(19) United States
(12) Patent Application Publication
(10) Pub. No.: US 2025/0304895 A1
(43) Pub. Date: Oct. 2, 2025

(54) MICROFLUIDIC DEVICE FOR INDUCING BIDIRECTIONAL OSCILLATORY SHEAR STRESS ON BIOLOGICAL CELLS

(71) Applicant: Indian Institute of Science, Bangalore (IN)

(72) Inventors: Namrata Gundiah, Bangalore (IN); Jaywant H. ARAKERI, Bangalore (IN); Suyog V. MAHULKAR, Bangalore (IN); Neha PADDILLAYA, Bangalore (IN)

(21) Appl. No.: 18/617,382

(22) Filed: Mar. 26, 2024

(30) Foreign Application Priority Data
Jan. 27, 2023 (IN) 202341005523

(51) Int. Cl. C12M 3/06; C12M 1/00; C12M 1/34; C12M 1/42

(52) U.S. Cl. CPC C12M 23/16; C12M 23/38; C12M 35/04; C12M 41/36

(57) ABSTRACT

The present disclosure discloses a microfluidic device (100) for inducing oscillatory bi-directional shear stress on biological cells (10). The device (100) includes a coverslip (1) to receive a plurality of biological cells (10). A cover member (2) is disposed on the coverslip (1) and defines a chamber (6). The cover member (2) includes a first inlet section (3) and a second inlet section (4) to selectively receive and channelize fluid at a first and a second predetermined velocity into the chamber (6). The fluid channelized at the first predetermined velocity and the second predetermined velocity into the chamber (6) creates a predefined oscillatory bi-directional flow pattern to induce predefined wall shear stress on the plurality of biological cells (10). The configuration of the microfluidic device facilitates study of vascular biology by generating controlled flow dynamics that mimic the changes in wall shear stress in the monolayers of biological cells (10).

### TECHNICAL FIELD

The present disclosure relates in general to a field of biomedical sciences. Particularly, but not exclusively, the present disclosure relates to subjecting controlled bidirectional oscillatory shear stress on biological cells using a microfluidic device.

### BACKGROUND OF DISCLOSURE

Generally, the pathophysiology of arterial disease is highly complex and primarily involves dysfunction of the biological cell monolayer, which lines an inner layer of blood vessels. Typically, mechanical forces are regulators of vascular biochemistry and gene expression and produce varied effects on the cellular mechano-transduction processes and tissue homeostasis. The cellular responses to mechanical stimuli are important during angiogenesis, vascular remodeling, and atherosclerosis. The endothelial cells are generally exposed to cyclically varying, oscillatory, and sometimes high shear stresses due to the flowing blood. The earliest lesions of atherosclerosis generally develop in arterial bifurcations and along curved regions. Cell morphologies are spindle-shaped and aligned in the flow direction under laminar unidirectional flow conditions. However, the biological cells are round and do not have a uniform orientation in regions with turbulent flows. The production of inflammatory markers, such as reactive oxygen species (ROS), increases due to oscillatory shear stresses, whereas laminar flows inactivate cellular inflammation.

With the advent of technology, many devices have been developed to study vascular biology, which uses laminar unidirectional flows. For example, several such devices are microfluidic devices, cone-plate viscometers, and orbital shakers. Additionally, more devices have been developed that utilize lamellar flows to assess changes to the mechanobiological processes. The conventional devices use straight channels with laminar or oscillatory stimuli to mimic the in vivo cellular milieu. However, physiological flows through arterial vessels are highly complex due to unsteady or pulsatile and spatially developing flow conditions. Furthermore, the arterial vessels are defined with high curvature, resulting in secondary flows caused by an imbalance between centrifugal forces and the radial pressure gradients. Interactions between flow unsteadiness and wall curvature are highly nonlinear and result in various vortical structures, flow reversals, flow separations, and wall shear stress stagnation/fixed points in arteries. Thus, the conventional devices to study vascular biology fail to generate controlled flow dynamics that mimic the changes in wall shear stress (WSS) as seen in-vivo on the monolayers of endothelial cells, which is of primary importance in understanding the mechanistic reasons underlying arterial disease.

The present disclosure is directed to overcome one or more limitations stated above.

### SUMMARY OF THE DISCLOSURE

One or more shortcomings of the conventional system or method for inducing shear stress on biological cells for analysis are overcome, and additional advantages are provided through the microfluidic device, as claimed in the present disclosure. Additional features and advantages are realized through the techniques of the present disclosure. Other embodiments and aspects of the disclosure are described in detail herein and are considered a part of the claimed disclosure.

In a non-limiting embodiment of the present disclosure, a microfluidic device for inducing bidirectional oscillatory shear stress on biological cells. The microfluidic device includes a coverslip which is defined with a flow surface adapted to receive a plurality of biological cells. Further, the microfluidic device includes a cover member that is disposed on the coverslip. The cover member and the coverslip define a chamber for receiving fluid. The cover member further comprises a first inlet section that is defined at a portion of the cover member, where the first inlet section is configured to selectively receive and channelize fluid at a first predetermined velocity into the chamber. Furthermore, a second inlet section is defined at a portion of the cover member away from the first inlet section, where the second inlet section is configured to selectively receive and channelize fluid at a second predetermined velocity into the chamber. The fluid channelized at the first predetermined velocity and the second predetermined velocity into the chamber creates a predefined oscillatory bi-directional flow pattern to induce predefined wall shear stress on the plurality of biological cells. The configuration of the microfluidic device facilitates the study of vascular biology by generating controlled flow dynamics that mimic the changes in wall shear stress in the monolayers of biological cells to understand the mechanistic reasons underlying arterial disease.

In an embodiment, the plurality of biological cells are cultured on the coverslip.

In an embodiment, the microfluidic device includes an outlet section that is defined at a portion of the cover member that is opposite to the first inlet section and the second inlet section. The outlet section is configured to dispense fluid out of the chamber. Additionally, the outlet section is fluidly coupled to a reservoir which is adapted to receive and store fluid from the chamber.

In an embodiment, the first inlet section, the second inlet section and the outlet section are defined in a spaced apart configuration with an angular separation.

In an embodiment, the coverslip is made of a transparent material for microscopic observation of the plurality of biological cells.

In an embodiment, the coverslip and the cover member are adapted to be positioned within a frame configured to encompass the microfluidic device. The frame includes a first part defined with a cavity to receive the microfluidic device and a second part adapted to be fixed on the first part to enclose the microfluidic device.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE ACCOMPANYING DRAWINGS

The novel features and characteristic of the disclosure are set forth in the appended claims. The disclosure itself, however, as well as a preferred mode of use, further objectives and advantages thereof, will best be understood by reference to the following detailed description of an illustrative embodiment when read in conjunction with the accompanying figures. One or more embodiments are now described, by way of example only, with reference to the accompanying figures wherein like reference numerals represent like elements and in which:

FIG. 1 illustrates a perspective view of a microfluidic device for inducing bidirectional oscillatory shear stress on biological cells, according to an embodiment of the present disclosure.

FIG. 2 illustrates a top view of the microfluidic device, according to an embodiment of the present disclosure.

FIG. 3 illustrates a perspective view of a coverslip of the microfluidic device cultured with a plurality of biological cells, according to an embodiment of the present disclosure.

FIG. 4 illustrates a perspective view of a chamber, according to an embodiment of the present disclosure.

FIG. 5 illustrates a graphical representation of the temporal variations in wall shear stress induced at given point within the chamber, according to an embodiment of the present disclosure.

FIG. 6a illustrates a graphical representation of a match between desired circular shear rosette and computationally replicated shear rosette, according to an embodiment of the present disclosure.

FIG. 6b illustrates a graphical representation of predetermined velocity at first inlet section and second inlet section, according to an embodiment of the present disclosure.

The figures depict embodiments of the disclosure for purposes of illustration only. One skilled in the art will readily recognize from the following description that alternative embodiments of the structures and methods illustrated herein may be employed without departing from the principles of the disclosure described herein.

### DETAILED DESCRIPTION

While the embodiments in the disclosure are subject to various modifications and alternative forms, specific embodiments thereof have been shown by way of example in the figures and will be described below. It should be understood, however, that it is not intended to limit the disclosure to the particular forms disclosed, but on the contrary, the disclosure is to cover all modifications, equivalents, and alternatives falling within the scope of the disclosure.

It is to be noted that a person skilled in the art would be motivated by the present disclosure and modify various features of the microfluidic device without departing from the scope of the disclosure. Therefore, such modifications are considered to be part of the disclosure. Accordingly, the drawings show only those specific details that are pertinent to understanding the embodiments of the present disclosure so as not to obscure the disclosure with details that will be readily apparent to those of ordinary skill in the art having the benefit of the description herein. Also, the microfluidic device of the present disclosure may be employed to test pharmacological treatments, but not limited to, such as those for hypertension, β-blockers, diabetes, and assess interactions of bacterial/viral cells with endothelial cells, thrombosis development, and deep vein thrombosis, among others.

The terms “comprises”, “comprising”, or any other variations thereof used in the disclosure, are intended to cover a non-exclusive inclusion, such that the device that comprises a list of components does not include only those components, but may include other components not expressly listed or inherent to such device or apparatus. In other words, one or more elements in the device proceeded by “comprises . . . a” does not, without more constraints, preclude the existence of other elements or additional elements in the device.

Henceforth, the present disclosure is explained with the help of figures illustrating a microfluidic device. However, such exemplary embodiments should not be construed as limitations of the present disclosure. A person skilled in the art can envisage various such embodiments without deviating from the scope of the present disclosure.

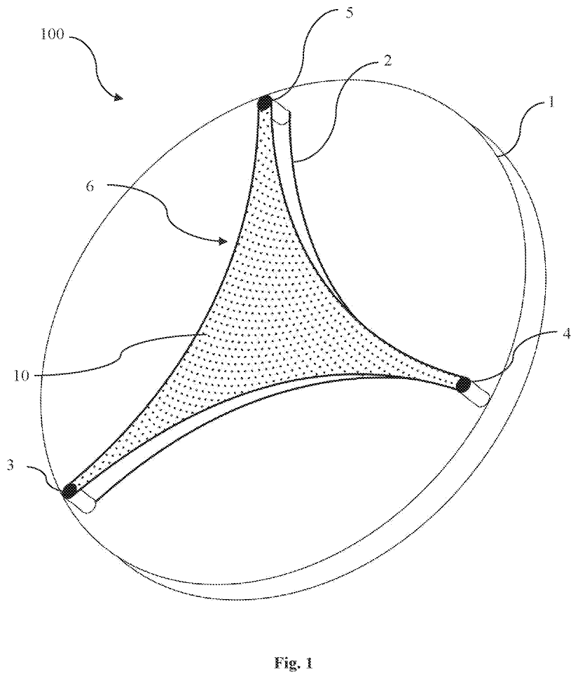

Reference will now be made to the exemplary embodiments of the disclosure, as illustrated in the accompanying drawings. Wherever possible, same numerals have been used to refer to the same or like parts. The following paragraphs describe the present disclosure with reference to FIGS. 1-6b.

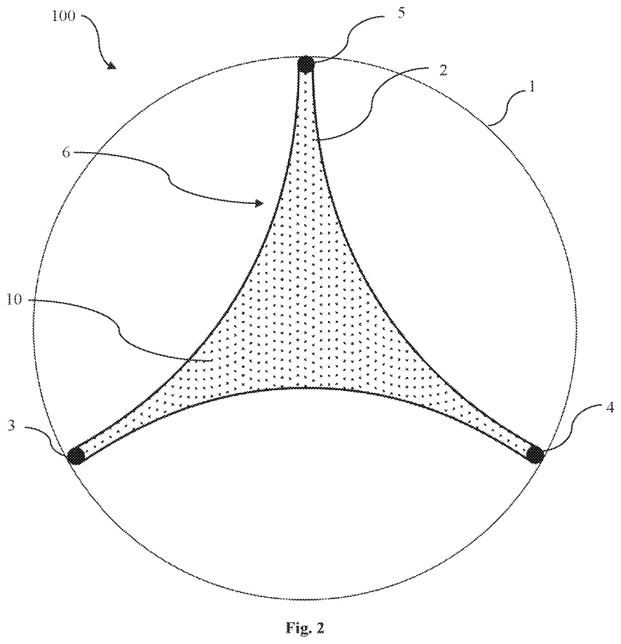

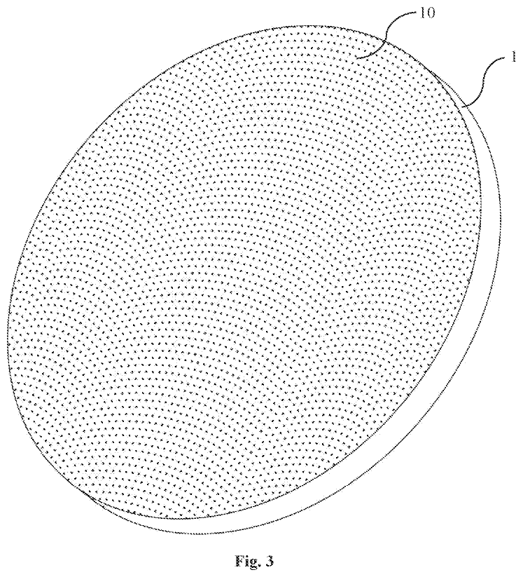

FIGS. 1 and 2, are exemplary embodiments of the present disclosure, illustrating a microfluidic device (100) [hereafter interchangeably referred to as “device (100)”] that may be configured to induce bidirectional oscillatory shear stress on biological cells (10). In an embodiment, the microfluidic device (100) may function as a body on chip device (100) or as an organ on chip device (100) which may be capable of emulating human physiological response to fluids or drugs and may have potential to capture both efficacy of the fluid or the drug. In an embodiment, the biological cells (10) may be endothelial cells. The device (100) may include a coverslip (1) which may be defined with a flow surface to receive a plurality of biological cells (10). In an embodiment, the plurality of biological cells (10) may be cultured on the coverslip (1) or a cover slip [as seen in FIG. 3]. In an embodiment, the plurality of biological cells (10) that may be cultured on the coverslip (1) may be defined with predefined shape and dimensions based on the requirement. The cultured plurality of biological cells (10) is formed as a monolayer over the flow surface. Further, the coverslip (1) may be made of a transparent material which may aid in the microscopic observation of the plurality of biological cells (10). Additionally, the device (100) may include a cover member (2) [as seen in FIG. 4]. The cover member (2) may be adapted to be disposed on the coverslip (1). In an embodiment, the cover member (2) may be disposed on the coverslip (1) such that the plurality of biological cells (10) received on the flow surface of the coverslip (1) may be covered. In an embodiment, the cover member (2) that may be positioned on the coverslip (1) may define a chamber (6). The chamber (6) may be configured to receive a fluid such that the fluid may interact with the plurality of biological cells (10) on the coverslip (1). In an illustrated embodiment, the first inlet section (3), the second inlet section (4) and the outlet section (5) in the spaced apart configuration resemble a lambda profile. However, this should not be considered as a limitation as the chamber (6) may be defined with any profile which enable the fluid to interact with the plurality of biological cells (10) to induce the bidirectional oscillatory shear stress.

In an embodiment, the cover member (2) is made of a polymeric material. For example, the cover member (2) may be made of a poly dimethyl siloxane (PDMS) material, glass, acrylic and the like or any other material suitable for forming the chamber (6) and allowing fluid flow and fluid interaction with the plurality of biological cells (10).

Further referring to FIGS. 1, 2 and 4, the cover member (2) may be defined with a first inlet section (3) which may be defined as a portion of the cover member (2). The first inlet section (3) may be configured to selectively receive and channelize fluid at a first predetermined velocity into the chamber (6). Furthermore, the cover member (2) may be defined with a second inlet section (4) which may be defined as a portion of the cover member (2). The second inlet section (4) may be configured to selectively receive and channelize fluid at a second predetermined velocity into the chamber (6). Fluid channelized at the first inlet section (3) and the second inlet section (4) at the first predetermined velocity and the second predetermined velocity, respectively, into the chamber (6) may create a predefined flow pattern in the chamber (6) to induce shear stress on the biological cells (10). In an embodiment, temporal variation of wall shear stress (WSS) induced at any given point in the chamber (6) may be called a shear rosette pattern which may be defined with any regular or irregular shape, that is, any complex realistic shear rosettes may be induced on the plurality of biological cells (10). The effects of the wall shear stress on the plurality of biological cells (10) may be observed by an operator or a lab technician. In an embodiment, the first inlet section (3) and the second inlet section (4) may be fluidly connected to a first pump and a second pump, respectively. In an embodiment, the first pump and the second pump may be syringe pumps. The first pump may be configured to supply fluid into the first inlet section (3) at the first predefined velocity, and the second pump may be configured to supply fluid into the second inlet section (4) at the second predefined velocity.

Further, the cover member (2) of the device (100) may be defined with an outlet section (5). The outlet section (5) may be defined as a portion of the cover member (2) that may be opposite to the first inlet section (3) and the second inlet section (4). The outlet section (5) may be configured to dispense fluid out of the chamber (6). Furthermore, the outlet section (5) may be fluidly coupled to a reservoir adapted to receive and store fluid from the chamber (6).

In an illustrated embodiment, the first inlet section (3), the second inlet section (4) and the outlet section (5) may be defined in a spaced apart configuration with an angular separation. This spaced apart configuration of the first inlet section (3) and the second inlet section (4) aids in creating the predefined bidirectional oscillatory flow pattern. Additionally, the chamber (6) may be defined with any number of inlet and outlet sections which may aid in introducing the fluid into and out of the chamber (6) for inducing bidirectional oscillatory shear stress on the plurality of biological cells (10).

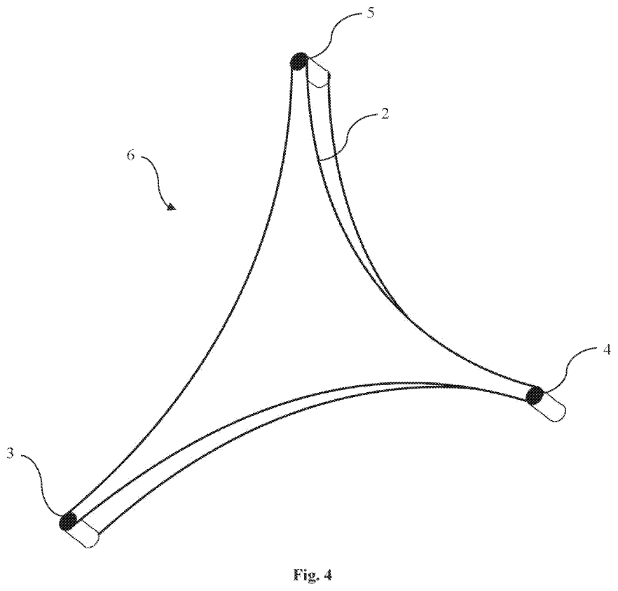

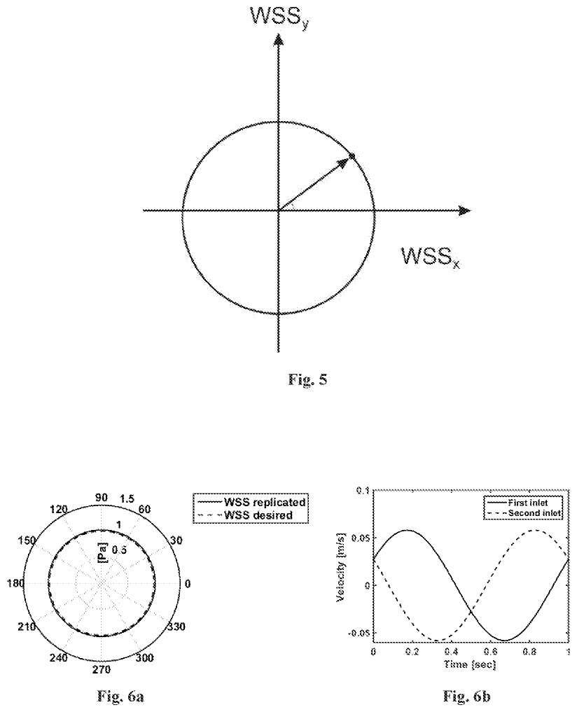

In an embodiment, the first predetermined velocity and the second predetermined velocity may be determined based on the predefined oscillatory bi-directional flow rates to generate a number of different flow conditions. For example, the first predetermined velocity and the second predetermined velocity may be determined based on the required shear rosette pattern [as seen in FIG. 5] of the fluid to be induced on the plurality of biological cells (10). In an embodiment, the predefined flow pattern may be selected based on the flow pattern of fluid, which may be required to be replicated to study the effects of the fluid flow over the plurality of biological cells (10). For example, the flow pattern of fluid within the arterial vessels may be replicated within the chamber (6) of the device (100), such that the effects of such flow pattern may be studied.

In an embodiment, the first predetermined velocity and the second predetermined velocity may be determined by a laminar and a quasi-steady assumption to produce a predefined flow pattern. For example, the predefined wall shear stress (WSS) pattern may resemble a rosette shape. In an embodiment, the required rosette shape may be created in the central region of the chamber (6). The centroid of the chamber (6) may be used as the origin of the coordinate system. Flow velocities at each of the two inlets, labeled as 1 and 2, are given by:

$${V}_{1} = {V}_{o} cos ( η ) {V}_{2} = {V}_{o} sin ( η ) \qquad ( 1 )$$

Vₒ is the amplitude, and η is the phase angle of the inlet velocity into the chamber (6). The wall shear stress (WSS) (=6 μū/h) for fully developed fluid flows depends on the dynamic viscosity, μ, the height of the channel, h, and the mean flow velocity, ū. The vector addition of velocities from both inlets at the centroid of the chamber (6) is used to calculate the WSS in the X and Y directions in the chamber (6). The ratio of WSS components at the centroid is given by:

$$\frac{{WSS}_{X}}{{WSS}_{Y}} = ( \frac{{C}_{Y}}{{C}_{X}} \frac{cos ( η ) - sin ( η )}{cos ( η ) + sin ( η )} \frac{1}{tan ( π / 3 )} ) \qquad ( 2 )$$

The ratio of WSS components in the X and Y directions is required to obtain a flow profile pattern specific to resemble the desired shear rosette shape. An increase in the cross-sectional area of the inlet arm from the inlet port to the centroidal region reduces the flow velocity. Cx and Cy are the embodiment-specific coefficients that account for increase in cross-section area of a portion of the chamber (6) from the one of the inlet section to the centroidal region. Substituting WSS values at each instant t, and area increment coefficients in equation (2), the expression for η can be evaluated. The magnitude of the velocity Vₒ in terms of WSS can be given by equation (3).

$${V}_{o} = \frac{h {WSS}_{X}}{6 μ} \frac{{C}_{X}}{( cos ( η ) - sin ( η ) ) cos ( π / 3 )} \qquad ( 3 )$$

The solution to the both equation (2) and equation (3) may predict the input velocity at the inlet section for each time step independently (quasi-steady approach), whose functional form is given by equation (1). Further sinusoidal fit is used to correlate velocity as the function of time: V₁=V₁(t) and V₂=V₂(t).

For example, a circular shear rosette with a magnitude of WSS as 1 Pa, which is a typical value for biological cells (10) (for example, endothelial cells), the normal inlet velocities [as seen in FIG. 6b] are given by:

$${V}_{1} = 0.0 \; 579 sin ( 2 π t + 0.4 \; 92 ) \qquad ( 4 )$$

$${V}_{2} = 0.0 \; 579 sin ( 2 π t + 2.649 )$$

For the given velocities, computation has been performed using suitable computing means, such as but not limited to, computational fluid dynamics. The computationally obtained shear rosette at the centroid of the chamber (6) shows an exact match with the desired circular rosette, [as seen in FIG. 6a]. This validates the analytical model. The validation is not limited to circular shape, it is useful for input flow velocity calculation for any irregular shape as desired. For example, the input flow velocity may be calculated for any geometric shape which may aid in inducing bidirectional oscillatory shear stress on the plurality of biological cells (10). That is, any other complex realistic rosettes may be created for inducing the bidirectional shear stress on the plurality of biological cells (10).

Further, in an embodiment, the device (100) having the coverslip (1) and the cover member (2), may be positioned within a frame [not shown in Figs] which can encompass the device (100) in the center which may be adapted to seal the device (100) temporarily during the experiment and position the device (100) under a microscope. The frame may include a first part which may be defined with a cavity to receive the device (100). Further, the frame may include a second part which may be fixed on the first part to enclose the device (100). Additionally, the second part of the frame may include a hole that may be configured to facilitate the operator to observe the biological cells (10) through the coverslip (1). The frame may be adapted to apply uniform pressure on the cover member (2) on the coverslip (1) to form an air-tight seal. The frame may not be limited to a specific shape of the device (100) and can be used for any device (100) having any geometrical shape of the cover member (2). The frame may be removed after the experiment to collect the coverslip (1) with the plurality of biological cells (10) for staining and analysis. In an embodiment, the frame may be re-usable such that the frame may be used for any number of experiments.

In an operational embodiment, the device (100) may be employed as a body on chip device (100) which may be employed in order to observe the effects of the wall shear stress on the biological cells (10). Initially, the plurality of biological cells (10) may be cultured on the coverslip (1). The coverslip (1) having the cultured biological cells (10) may be covered by the cover member (2) to form the chamber (6). The device (100) having the coverslip (1) and the cover member (2), may be positioned within a frame which can encompass the microfluidic device (100) in the center to seal it temporarily during the experiment. Upon forming the chamber (6), the first inlet section (3) and the second inlet section (4) may be fluidly connected to the first syringe pump and the second syringe pump, respectively. Fluid from the first syringe pump and the second syringe pump may be introduced into the chamber (6) at the first predetermined velocity and the second predetermined velocity, respectively. Fluid introduced into the chamber (6) at the first predetermined velocity and the second predetermined velocity may form the predefined flow pattern. The predefined flow pattern may be based on the required shear rosette on the biological cells (10) that may replicate the flow pattern within arterial vessels. Fluid in the predefined flow pattern may exert wall shear stress on the biological cells (10) cultured on the coverslip (1), which may lead to the displacement of the biological cells (10). The displacement of the biological cells (10) may be observed by the operator through the microscope. The frame may be then removed after the experiment to collect the coverslip (1) with the biological cells (10) for staining and analysis.

In an embodiment, biological cells (10) from different individuals in the device (100) may be envisioned to target individualized therapies to treat arterial disease.

In an embodiment, the microfluidic device (100) facilitates monitoring the effects of wall shear stress on the biological cells (10).

In an embodiment, the microfluidic device (100) allows for growing the biological cells (10) on the coverslip (1) to form a monolayer before clamping the cover member (2) to the coverslip (1) using the frame. The frame is not specific to the shape of the microfluidic chamber (6) and can be used for any microfluidic chamber (6) shape. The frame can be removed after the experiment and reused for other experiments.

In an embodiment, the device (100) facilitates controlled disturbed flows of fluid within the chamber (6). Further, the device (100) may be configured to exert controlled bidirectional oscillatory flows within the chamber (6) over the biological cells (10). The wall shear stress exerted on the biological cells (10) replicates changes in the endothelial mechanobiology. The observation of the changes in the biological cells (10) upon reaction to fluid in the predefined flow pattern facilitates an understanding of the various factors that may be involved in the pathophysiology of vascular disease. Furthermore, the reaction of the biological cells (10) to the predefined flow pattern of fluid may help determine pharmacological treatments to help identify and assess vascular disease treatments such as for atherosclerosis, deep venous thrombosis, and aneurysms.

In an embodiment, the device (100) facilitates the generation of physiological and pathophysiological flows, representative of curved arterial regions, bifurcations and near obstructions, which are essential to assess cellular changes and gene expression levels of various protein markers. The device (100) may also be employed to test pharmacological treatments, such as those for β-blockers for hypertension, diabetes, and assess interactions of bacterial/viral cells with endothelial cells, thrombosis development, deep vein thrombosis and the like.

In an embodiment, the device (100) permits investigations into the biological cell mechanisms for drug efficacy using human cells, which can reduce trials with animal studies.

It should be imperative that the microfluidic device (100) and any other elements described in the above detailed description should not be considered as a limitation with respect to the figures. Rather, variations to such systems and methods should be considered within the scope of the detailed description.

### EQUIVALENTS

With respect to the use of substantially any plural and/or singular terms herein, those having skill in the art can translate from the plural to the singular and/or from the singular to the plural as is appropriate to the context and/or application. The various singular/plural permutations may be expressly set forth herein for the sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation, no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general, such construction is intended in a sense that one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general, such construction is intended in a sense that one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

While various aspects and embodiments have been disclosed herein, other aspects and embodiments will be apparent to those skilled in the art. The various aspects and embodiments disclosed herein are for purposes of illustration and are not intended to be limiting, with the true scope being indicated by the following claims.

### REFERRAL NUMERALS

| Reference Number | Description |
|---|---|
| 100 | Device |
| 1 | Coverslip |
| 2 | Cover member |
| 3 | First inlet section |
| 4 | Second inlet section |
| 5 | Outlet section |
| 6 | Chamber |
| 10 | Biological cell |

We claim:

1. A microfluidic device (100) for inducing bidirectional oscillatory shear stress on biological cells (10), the microfluidic device (100) comprising:
a coverslip (1), defined with a flow surface adapted to receive a plurality of biological cells (10); and
a cover member (2) disposed on the coverslip (1), wherein the cover member (2) and the coverslip (1) define a chamber (6) for receiving fluid, characterized in that, the cover member (2) comprises:
a first inlet section (3) defined at a portion of the cover member (2), wherein the first inlet section (3) is configured to selectively receive and channelize fluid at a first predetermined velocity into the chamber (6); and
a second inlet section (4) defined at a portion of the cover member (2) away from the first inlet section (3), wherein the second inlet section (4) is configured to selectively receive and channelize fluid at a second predetermined velocity into the chamber (6);
wherein, fluid channelized at the first predetermined velocity and the second predetermined velocity into the chamber (6) creates a predefined oscillatory bi-directional flow pattern to induce predefined wall shear stress on the plurality of biological cells (10).

2. The microfluidic device (100) as claimed in claim 1, wherein the plurality of biological cells (10) are cultured on the coverslip (1).

3. The microfluidic device (100) as claimed in claim 1, comprises an outlet section (5) defined at a portion of the cover member (2) opposite to the first inlet section (3) and the second inlet section (4), the outlet section (5) is configured to dispense fluid out of the chamber (6).

4. The microfluidic device (100) as claimed in claim 3, wherein the outlet section (5) is fluidly coupled to a reservoir adapted to receive and store fluid from the chamber (6).

5. The microfluidic device (100) as claimed in claim 1, wherein the first inlet section (3), the second inlet section (4) and the outlet section (5) are defined in a spaced apart configuration with an angular separation.

6. The microfluidic device (100) as claimed in claim 1, wherein the coverslip (1) is made of a transparent material for microscopic observation of the plurality of biological cells (10).

7. The microfluidic device (100) as claimed in claim 1, wherein the coverslip (1) and the cover member (2) are adapted to be positioned within a frame configured to encompass the microfluidic device (100).

8. The microfluidic device (100) as claimed in claim 7, wherein the frame includes a first part defined with a cavity to receive the microfluidic device (100) and a second part adapted to be fixed on the first part to enclose the microfluidic device (100).

9. A microscope configured to selectively receive the microfluidic device (100), as claimed in claim 1, to observe reaction of a plurality of biological cells (10) to bi-directional oscillatory flow pattern.
